(19) United States
(12) Patent Application Publication
(10) Pub. No.: US 2009/0229346 A1
(43) Pub. Date: Sep. 17, 2009

(54) Systems and method for capture substrates

(76) Inventors: Daniel Alvarez, JR., San Diego, CA (US); Troy B. Scoggins, San Diego, CA (US); Jeffrey J. Spiegelman, San Diego, CA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/989,553

(22) PCT Filed: Jul. 31, 2006
(86) PCT No.: PCT/US2006/029670

§ 371 Date: Sep. 26, 2008

Related U.S. Application Data
(60) Provisional application No. 60/704,792, filed on Aug. 2, 2005.

(51) Int. Cl. G01N 33/00; B01D 59/26

(52) U.S. Cl. 73/28.04; 95/90; 95/148

(57) ABSTRACT

A method of detecting a molecular species in an electronics processing environment is disclosed. The method exposes a capture substrate to the processing environment. The capture substrate has a surface area different from the surface area of an electronic substrate undergoing electronics processing. The molecular species is transferred from the environment to the capture substrate. A characteristic of the molecular species is identified, thereby detecting the species. Other methods utilize a capture substrate to remove the molecular species from an electronic processing environment, or use the capture substrate to determine the presence of a molecular species in a transfer container operating between two process environments or two intermediate process steps. Systems for carrying out the methods are also disclosed.

### RELATED APPLICATION

This application claims benefit of U.S. Provisional Application 60/704,792, filed Aug. 2, 2005. The entire teachings of the above application are incorporated herein by reference.

### BACKGROUND OF THE INVENTION

Real time information regarding the presence of molecular contamination is becoming of increasing importance during electronics manufacturing. With the increasing expense and time of device processing, accurate information regarding the state of a processed substrate upon completion of an intermediate step would be advantageous. Especially important is the need for molecular species information, as opposed to just the general morphology of a substrate, since such contamination may not manifest its presence until several subsequent steps are performed beyond the initial point of contamination. As well, molecular species need to be detected at lower and lower concentration levels as device feature sizes continue to get smaller. Devices undergoing electronics processing are especially susceptible to contamination during transfer of the devices between intermediate processes. Transfer containers, such as front opening unified pods, may inadvertently introduce contaminants to materials being transferred therein through leakage or off-gassing of the container's construction.

### SUMMARY OF THE INVENTION

Embodiments of the invention are drawn to methods and systems of utilizing a capture substrate to purify an environment and/or identify the presence of a molecular species in the environment. Such embodiments are particularly advantageous when the environment is within a transfer container utilized in electronics manufacturing, potentially allowing the purification and/or identification of molecular species that are contaminants within and between individual processing steps during real time device manufacturing. Unlike witness wafers, which have been used in model experiments to derive general information regarding contamination in a hypothetical working tool environment, some embodiments of the invention discussed herein allow the analysis of real time information to determine process contamination as an actual process is being performed.

One embodiment of the invention is directed to a method of detecting a molecular species in an environment for electronics processing of an electronic substrate. A capture substrate, having a surface area different from the electronic substrate, is exposed to an electronics processing environment having the molecular species. The molecular species is transferred to the capture substrate. A characteristic of the transferred molecular species is subsequently identified, thereby detecting the molecular species.

The capture substrate may comprise silicon, a low k dielectric, copper, or a surface that mimics the surface characteristics of the electronic substrate undergoing electronics processing. The molecular species may be a contaminant. The environment may comprise a flowing fluid or a substantially quiescent fluid. The environment may be within a transfer container, preferably a front opening unified pod (FOUP). The FOUP may be configured to hold at least 26 wafer-shaped substrates. The FOUP may contain 25 wafers undergoing electronics processing and a capture substrate. The electronic substrate is preferably a silicon wafer and more preferably an unprocessed single crystal silicon wafer. The capture substrate has a surface area different from the electronic substrate, for example, the capture substrate may have a surface area at least about 10 times the surface area of the silicon wafer. Preferably, the capture substrate has a surface area at least about 25 times the surface area of the silicon wafer. More preferably, the capture substrate has a surface area of at least about 100 times the surface area of the silicon wafer. Transfer of the molecular species to the capture substrate may also purify the environment of the molecular species. The characteristic of the molecular species may be identified in part by desorbing the species from the capture substrate.

Another embodiment of the invention is directed to removing a molecular species from an environment for electronics processing of an electronic substrate. A capture substrate, having a surface area different from the electronic substrate, is exposed to an environment having the molecular species. The molecular species is transferred to the capture substrate, thereby removing the molecular species from the environment.

In another embodiment of the invention, a system for diagnosing the presence of a species in an environment for electronics processing of an electronic substrate is presented. The system includes a transfer container that encloses an environment, and a capture substrate contained within the transfer container. The capture substrate has a surface area different from the electronic substrate. The system may further include a thermal desorption device located in a minienvironment configured to remove a molecular species from the capture substrate when the substrate is mounted in the thermal desorption device.

In another embodiment of the invention, a method of determining the presence of a molecular species in a transfer container operating between two minienvironments is presented. A capture substrate is loaded from a first minienvironment into a transfer container, the container also holding at least one electronic substrate from the first minienvironment. The transfer container is transported from the first minienvironment to a second minienvironment. The capture substrate is removed and analyzed to determine the presence of at least one molecular species. Optionally, the molecular species is subsequently removed from the capture substrate and reutilized in a transfer container during a subsequent transfer to another minienvironment.

Another embodiment of the invention is directed to a method of determining the presence of a molecular species in a transfer container operating in an electronics manufacturing process. At least one processing step is completed in an electronics manufacturing process utilizing a plurality of steps. A transfer container is loaded with a capture substrate, and holds at least one electronic substrate processed during a previous processing step. The transfer container is transported to a location to perform a subsequent processing step. The capture substrate is removed and analyzed for the presence of the molecular species. The method may be repeated as subsequent process steps are performed.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 presents a schematic diagram of a plurality of processes utilized in an electronics processing fab that includes tool environments, minienvironments with a robot and desorption unit, and a front opening unified pod for transferring processed substrates between two processes, in accordance with embodiments of the invention.

FIG. 2 presents a schematic of a front opening unified pod for holding 26 wafer-shaped substrates, in accordance with an embodiment of the invention.

FIG. 3 presents a desorption unit for use with embodiments of the invention to analyze/identify a molecular species transferred to a capture substrate.

### DETAILED DESCRIPTION OF THE INVENTION

Embodiments of the invention are drawn to methods and systems of utilizing a capture substrate to purify an environment and/or identify the presence of a molecular species in the environment. Such embodiments are particularly advantageous when the environment is within electronics manufacturing fabrication processing. Such environments may be characterized as having a concentration of one or more particular molecular species that is below a designated level (e.g. having no greater than 100 parts per million of one or more molecular species on a volume basis).

In one embodiment of the invention, a method of detecting a molecular species in an environment in presented. A capture substrate is exposed to an environment that is used to manufacture or process an electronic substrate. The environment includes the molecular species to be detected. The environment can be an environment that is involved in the manufacturing or processing of the electronic substrate. The electronic substrate can be present or absent in the environment. For example, a capture substrate can be used in a tool environment to detect or remove a molecular species (e.g., a contaminant) when the electronic substrate is present or absent.

In accordance to the present invention, the electronic substrate can be an electronic device. In a preferred embodiment, the electronic substrate is a silicon wafer. More preferably, the electronic substrate is an unprocessed single crystal silicon wafer. The molecular species is transferred from the environment to the capture substrate. Subsequently, a characteristic of the molecular species, which is transferred to the capture substrate, is identified, thereby detecting the molecular species.

Such an embodiment may help identifying sources of contamination in a multistep processing environment, thereby preventing further downstream contamination. Processing a batch of contaminated substrates in a tool environment may result in contamination of the tool environment. Subsequent batches of the substrates be processed may also then be contaminated. By utilizing a capture substrate and identifying a characteristic of the contamination, materials in the transfer container may be disposed of before contaminating the tool environment. As well, contaminated substrates can be disposed of before undergoing potentially expensive downstream processing steps. In such a scenario, the contamination may occur in any of the process areas before the substrate is analyzed.

Exposing the capture substrate to an environment typically involves contacting at least a portion of a surface of the capture substrate with the environment. However, the capture substrate may also be completely surrounded by the environment, or exposed in any other manner. No specific time limitation is necessarily placed on the exposure, though it is enough to allow transfer of at least one molecular species in some embodiments of the invention.

In one embodiment of the present invention, the capture substrate has a surface area greater than the electronic substrate being processed or manufactured. Preferably, the electronic substrate is a silicon wafer. More preferably, the electronic substrate is an unprocessed single crystal silicon wafer. In one embodiment, the capture substrate has a surface area greater than the silicon wafer being processed or manufactured. Preferably, the capture substrate has a surface area at least about 10 times the surface area of the silicon wafer. More preferably, the capture substrate has a surface area at least about 25 times the surface area of the silicon wafer. Even more preferably, the capture substrate has a surface area at least about 100 times the surface area of the silicon wafer. In another specific embodiment, the capture substrate has a surface area greater than an unprocessed single crystal silicon wafer being processed or manufactured. Preferably, the capture substrate has a surface area at least about 10 times the surface area of the unprocessed single crystal silicon wafer. More preferably, the capture substrate has a surface area at least about 25 times the surface area of the unprocessed single crystal silicon wafer. Even more preferably, the capture substrate has a surface area at least about 100 times the surface area of the unprocessed single crystal silicon wafer. One skilled in the art can adjust the surface area of the capture substrate based upon the application and type of molecular species present. For example, when the capture substrate is used in the presence of x number of unprocessed single crystal silicon wafers, a silicon wafer with surface area of x times the surface area of the unprocessed single crystal wafer can be used as the capture substrate. The capture substrate will have the same capture capacity as the x number of unprocessed single crystal silicon wafers combined and will act as a sink for a molecular contaminant that binds to silicon surfaces.

Even though the capture substrate has a surface area different from that of the electronic substrate, the size of the capture substrate is preferred to be the same as the electronic substrate as a matter of convenience based upon the dimensions of the equipment(s) and container(s) used in the manufacturing process.

High surface area capture substrate can be generated by standard methods. For example, a porous silicon wafer can be etched to achieve high surface area and used as a capture substrate. The etching procedures and required equipments are well-known in the art.

The surface area generated can be determined by using a standard surface area determination technique (e.g., Langmuir isotherm method or Brunauer, Emmett, Teller (BET) method). The enhanced surface area of the capture substrate relative to the electronic substrate (e.g. a silicon wafer, an unprocessed silicon surface) allows for additional sites to which molecular species (e.g., contaminants) may reside, increasing the ability of the capture substrate to adsorb, bind, or associate with the molecular species.

High surface area capture substrates advantageously provide a high potential transfer area for holding molecular species. For example, if an unprocessed single crystal silicon wafer is in the presence of a capture substrate having a silicon surface area 25 times that of the unprocessed single crystal silicon wafer, the capture substrate essentially acts like 25 unprocessed silicon wafers in terms of total capture capacity in comparison to the unprocessed wafer. Thus, the capture wafer may act as a sink for a molecular contaminant that binds to silicon surfaces.

In accordance with the present invention, the surface area of the capture substrate can also be less than the surface area of the electronic substrate being processed. The capture substrate can be tailored for specific molecular species (e.g., contaminant(s)) in the environment. The capture substrate can be designed to comprise material(s) that have high capture capacity for the molecular species and thereby be more effective in capturing the molecular species than the electronic substrate undergoing electronics processing. Therefore, the capture substrate can have less surface area than the electronic substrate while still maintain high capture capacity for the molecular species. For example, capture substrate comprising a metal or metal oxide coating can be used for detecting or removing ammonia or base gases and acid gases. A coating of carbonaceous media or carbon nanotubes can be used to detect or capture hydrocarbon and refractory gases. One skilled in the art can readily determine the desired surface area of the capture substrate depending upon the nature of the molecular species and the capture substrate chosen.

Capture substrates are preferably utilized with at least one surface characteristic that is advantageous to its use. In accordance to the present invention, surface characteristic can represent material composition of the surface. Surface characteristic can also represent the way the surface interacts with the molecular species in the environment. In one particular embodiment, the capture substrate has a surface that mimics a surface characteristic of the substrate undergoing electronics processing. For example, in a silicon wafer processing environment, quality control of the silicon surface dictates identifying the presence or absence of particular molecular species on the surface. Thus, an appropriate capture substrate in this context is a silicon wafer or some type of substrate comprising silicon, such that the surface characteristics of a silicon wafer are mimicked to some degree. In another example, the capture substrate has a surface comprising copper. In particular, the presence of copper on a silicon surface of a capture substrate can promote the formation of time-dependent haze, which may act as a signature of contamination from acids or other contaminant species (see Münter, N. et al, “Formation of Time-Dependent Haze on Silicon Wafers,” Solid State Phenomena, Vol. 92 (2003) pp. 109-112). Other types of surface characteristics of capture substrates can also be tailored (e.g., low k dielectric material surface character).

Other types of surfaces on a capture substrate include surfaces that are tailored to attract a particular type of molecular species or contaminant (or a set of molecular species), regardless of the character of any other substrate being processed in the same environment. In such an embodiment, the capture substrate may act to help identify the presence of one or more particular molecular species and/or as a sink for the molecular species.

Capture substrates may be utilized in a variety of environments of an electronics manufacturing process. Examples of environments include environments enclosed within particular chambers of various processes or transfer containers used to transfer devices and substrates being worked upon between various processes. The particular environment may have a gas flowing through the environment (e.g., a front opening unified pod with a purge gas flowing through the container) or the environment may be substantially quiescent.

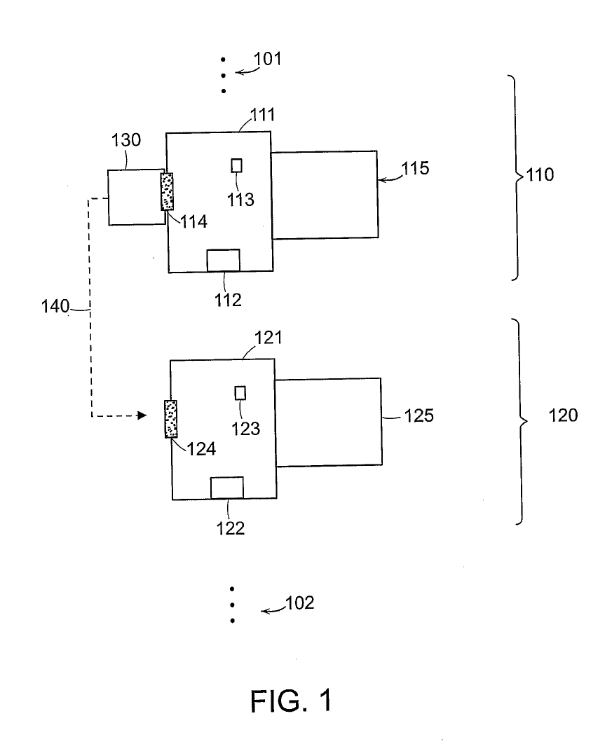

To illustrate some exemplary environments, an electronics manufacturing fab is typically comprised of a series of processes for performing various functions (e.g., etching substrates, applying masks, growing films, removing layers, forming features, etc.). As depicted schematically in FIG. 1, the various functions of a hypothetical fab are performed in a plurality of processes 110, 120, the ellipses 101, 102 indicating that the figure only depicts two adjacent intermediate processes of the entire manufacturing fab. Each process 110, 120 includes a tool environment 115, 125 and a corresponding minienvironment 111, 121. Minienvironments, also known as microenvironments, are typically enclosures that are built around process equipment. Minienvironments are typically integrated, controlled environments in production equipment where substrates reside and are separated from personnel and the general fab environment. One or more transfer containers 130, may connect to the minienvironment 111, 121 through a port 114, 124. Thus, capture substrates may be utilized in the tool environment 115, 125, a minienvironment 111, 121, and a transfer container 130 to detect or remove one or more molecular species from the respective environment.

A capture substrate is exposed to an environment within a transfer container, in a particular embodiment of the invention. Transfer containers utilized in electronics processing include, but are not limited to, standard mechanical interface pods (SMIF pods), front opening unified pods (FOUPs), front opening shipping boxes (FOSBs), stockers, isolation pods, and other containers for transporting wafers and/or electronic substrates. Transfer containers are typically utilized to transfer substrates, devices, or intermediate products thereof between process steps. Transfer containers may also be used to transport the finished products to remote locations, or raw materials, such as unprocessed silicon wafers, to the beginning processes of a fab.

Particular transfer containers, such as FOUPs, are non-hermetically sealed containers that may be susceptible to contamination since molecular species may leak into the container's enclosure. Furthermore, transfer containers may also include the use of plastics or elastomers that off-gas potential contaminants into the container enclosure. Utilization of a capture substrate within a transfer container that also holds electronic substrates or devices undergoing processing (e.g., silicon wafers) allows identification of the presence of a molecular species, which can result in the formation of damaged devices that materialize during downstream processing, as described earlier.

For example, as schematically diagrammed in FIG. 1, a FOUP 130 is loaded with silicon wafers that are processed in tool environment 115. The wafers are loaded into the FOUP 130 by a robot 113 working in the minienvironment 111. A capture substrate is also loaded into the FOUP 130. The FOUP 130 is closed and transported 140 to the next process 120 for further processing. During transport, the FOUP may hold the capture substrate and wafers for many hours until the next process and minienvironment are prepared to accept the FOUP's contents. Thus, contamination that the wafers in the FOUP are exposed to may be detected by examining the capture substrate while wafers are held in the FOUP or the minienvironment 121, before exposing the wafers to the next tool environment 125.

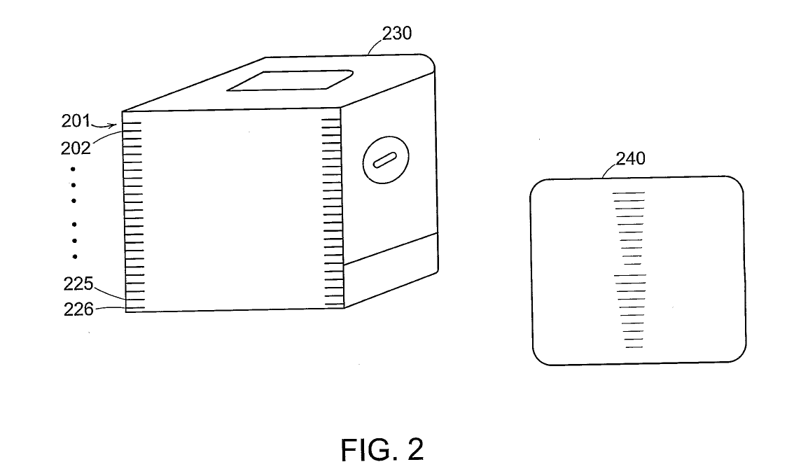

Particular embodiments of the invention utilize a FOUP configured to hold 26 or more wafer-shaped substrates. Typical FOUPs hold 25 silicon wafers for transport. As depicted in FIG. 2, a FOUP 200 comprises an enclosure 230 and a door 240. The FOUP enclosure 230 contains fixtures 201, 202, 225, 226 for holding 26 wafer shaped substrates. Typically, 25 silicon wafers undergoing electronics processing are held in 25 of the holding spots of the FOUP. The 26^{th} holding spot is reserved for a capture substrate. The dense packing of wafers shows that the addition of a place for an extra wafer-shaped substrate, such as a capture substrate, can be achieved without substantially altering the size of a typical FOUP. The 26 wafer FOUP allows a capture substrate to be incorporated into the FOUP for diagnostic/purifying purposes without changing the typical planning in fab processing on a basis of FOUPs holding 25 wafers.

Transfer of at least one molecular species from an environment to a capture substrate occurs without limitation to the mechanism of transfer. For example, the environment may be essentially quiescent, such that transfer of the molecular species from the environment to the capture substrate occurs primarily by diffusion (Fickian or non-Fickian in the case of a substrate having size features of the order of, or smaller than, the mean free path of the gas molecules in the environment). Alternatively, the environment may have a molecular species transferred by some other driving force besides a concentration gradient (e.g., a purge gas may flow through a FOUP enclosure containing the capture substrate). Furthermore, transfer of the molecular species does not provide a limitation on the interaction between the transferred molecular species and the capture substrate. Thus, upon transfer, the molecular species may be bound, adsorbed, or otherwise physically associated with the capture substrate. In some embodiments, the transferred molecular species is adsorbed to the capture substrate, and preferably to the substrate surface. In a related embodiment, the capture substrate may interact with the transferred molecular species to cause a reaction to occur with at least some of the molecular species (e.g., if the substrate acts as a catalyst).

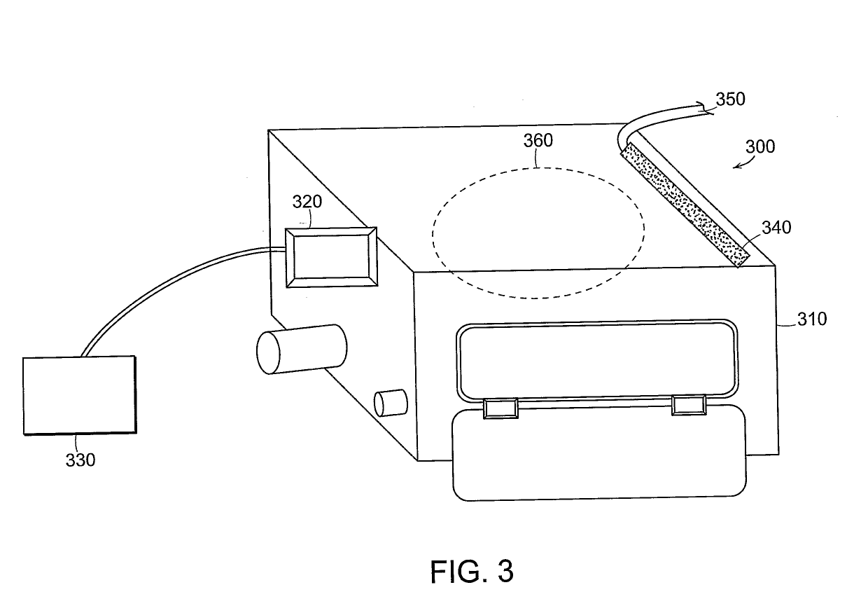

Identifying a characteristic of the molecular species that is transferred from the environment to the capture substrate may be performed using any of the techniques known to those skilled in the art. For example, desorption of the molecular species from the capture substrate may be performed using a thermal source, followed by subsequent analysis of the desorbed materials. As shown in FIG. 3, a desorption unit 300 is used to identify a molecular species on a capture substrate. The unit 300 has an air or nitrogen inlet 350 and a diffuser for the inlet gas 340. The unit 300 includes a substrate heater 360, which heats the substrate to desorb molecular species. Molecular species identification is performed with a Kamina e-nose 320 (see World Wide Web at www.specs.com/products/Kamina/Kamina.htm) hooked to a computer 330 to analyze the desorbed species with a gradient microchip array for gas analysis. Other identifying techniques, such as spectroscopic methods, may be utilized to characterize the molecular identity of the species. As well, desorption need not necessarily be used as part of the identification step.

In other embodiments of the invention, the use of capture substrates as described herein, which are exposed to an electronics manufacturing environment, also results in the removing a molecular species from the environment, thereby purifying the environment. In particular, the transfer of one or more molecular species from the environment to the capture substrate removes the molecular species from the environment, thus also purifying the environment. The environment may be purified to a particular concentration level with respect to one or more molecular species. As well, embodiments of the invention may also be directed to removing a molecular species from an electronics processing environment without regard to whether identification of the molecular species takes place. In an exemplary embodiment, an environment is exposed to a capture substrate. Transfer of a molecular species from the environment to the capture substrate thereby removes the molecular species from the environment. Thus, capture substrates may act as a purifier in some instances. The aforementioned embodiments may utilize any of the environments and any of the capture substrates described herein.

Related embodiments of the invention are directed to systems for diagnosing the presence of a molecular species, and/or purifying the presence of a molecular species, in an environment (e.g., an electronics manufacturing environment). The system includes a transfer container that encloses an environment and a capture substrate contained within the transfer container. In particular, the transfer container may have a surface area greater than an unprocessed single crystal silicon wafer. The capture substrate and the transfer container, however, may utilize any of the traits discussed herein regarding capture substrates or transfer containers.

Other embodiments of the invention are directed to determining the presence of a molecular species in a transfer container operating between at least two minienvironments. An exemplary, non-limiting, embodiment is schematically depicted in FIG. 1. The various functions of a hypothetical fab are performed in a plurality of processes 110, 120, the ellipses 101, 102 indicating that the figure only depicts two adjacent intermediate processes of the entire manufacturing fab. Each process 110, 120 includes a tool environment 115, 125 and a minienvironment 111, 121. Each minienvironment 111, 121 includes a robot 113, 123 for manipulating devices being processed. For example, a robot may load wafers out of a FOUP into a minienvironment and into a tool for processing. Upon completion of that process, the wafers may be withdrawn from the tool and placed into a transfer container, such as a FOUP, for transport to the next process tool. A capture substrate is included in the transfer container. For the processes 110, 120 shown in FIG. 1, each minienvironment 111, 121 includes a desorption unit 112, 122 (e.g., the unit shown in FIG. 3). Thus, when materials are transferred between two processes 110, 120, a capture substrate, present in the FOUP 130 used to transfer substrates undergoing processing, can be analyzed to determine the presence of a molecular species (e.g., contaminant) that may have contaminated the FOUP environment during transportation 140 of the FOUP 130.

Therefore, real-time information regarding the potential contamination of actual processed materials in a transfer container may be obtained to prevent downstream contamination of a tool, or prevent the expense of performing an expensive process on wafers or devices that are already defective. Any of the capture substrates or transfer containers described herein may be used with these embodiments.

In a related embodiment, the analyzed capture substrate may have the molecular species substantially removed after the capture substrate has been exposed to the FOUP environment. The capture substrate may then be reused in a subsequent transfer between two other processes. This allows the same capture substrate to be used over during transfers between processes.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of removing a molecular species from an environment for electronics processing of an electronic substrate, comprising:
providing a capture substrate, wherein the capture substrate does not have the same surface area as the electronic substrate;
exposing the capture substrate to the environment; and
transferring the molecular species from the environment to the capture substrate, thereby removing the molecular species from the environment.

2. The method of claim 1, wherein the electronic substrate is a silicon wafer.

3. The method of claim 2, wherein the silicon wafer is an unprocessed single crystal silicon wafer, which is undergoing electronics processing.

4. The method of claim 2, wherein the surface area of the capture substrate is greater than the silicon wafer.

5. The method of claim 4, wherein the surface area of the capture substrate is at least about 10 times the surface area of the silicon wafer.

6. The method of claim 4, wherein the surface area of the capture substrate is at least about 25 times the surface area of the silicon wafer.

7. The method of claim 4, wherein the surface area of the capture substrate is at least about 100 times the surface area of the silicon wafer.

8. The method of claim 1, wherein the capture substrate comprises silicon.

9. The method of claim 1, wherein the capture substrate comprises a low k dielectric.

10. The method of claim 1, wherein the capture substrate comprises copper and exposing the capture substrate includes exposing the copper to the environment.

11. The method of claim 1, wherein the capture substrate has a surface that mimics a surface characteristic of the electronic substrate.

12. The method of claim 1, wherein the environment is within a transfer container.

13. The method of claim 12, wherein the environment is within a front opening unified pod.

14. The method of claim 13, wherein the front opening unified pod is configured to hold at least 26 wafer-shaped substrates.

15. The method of claim 1, wherein the molecular species is a contaminant.

16. The method of claim 15, wherein transferring the molecular species thereby purifies the environment of the contaminant.

17. The method of claim 1, wherein the environment comprises a flowing fluid.

18. The method of claim 1, wherein the environment is substantially quiescent.

19. The method of claim 1 further comprising:
identifying a characteristic of the molecular species transferred to the capture substrate, thereby detecting the molecular species.

20. The method of claim 19, wherein identifying the characteristic of the molecular species comprises desorbing the species from the capture substrate.

21. A method of removing and detecting a molecular species in an environment for electronics processing of an electronic substrate, comprising:
providing a capture substrate, wherein the capture substrate does not have the same surface area as the electronic substrate;
exposing the capture substrate to the environment;
transferring the molecular species from the environment to the capture substrate; and
identifying a characteristic of the molecular species transferred to the capture substrate, thereby detecting the molecular species.

22. The method of claim 21, wherein identifying the characteristic of the molecular species comprises desorbing the species from the capture substrate.

23-39. (canceled)

40. A system for diagnosing the presence of a molecular species in an environment for electronics manufacturing of an electronic substrate, comprising:
a transfer container enclosing an environment; and
a capture substrate contained within the transfer container, wherein the capture substrate does not have the same surface area as the electronic substrate.

41. The system of claim 40 further comprising:
a thermal desorption device located in a minienvironment, wherein the thermal desorption device is configured to remove at least one molecular species from the capture substrate when the capture substrate is mounted in the thermal desorption device.

42-51. (canceled)

52. The system of claim 40, wherein the transfer container is a front opening unified pod.

53. The system of claim 52, wherein the front opening unified pod is configured to hold at least 26 wafer-shaped substrates.

54. The system of claim 52, wherein the front opening unified pod holds between 1 to 25 wafers undergoing electronics processing.

55. A method of determining the presence of a molecular species in a transfer container operating between at least two minienvironments, comprising:
a) loading a capture substrate from a first minienvironment into a transfer container, wherein the transfer container also holds at least one electronic substrate loaded from the first minienvironment;
b) transporting the transfer container from the first minienvironment to a second minienvironment;
c) removing the capture substrate from the transfer container; and
d) analyzing the capture substrate to determine the presence of the molecular species.

56. The method of claim 55 further comprising:
e) substantially removing the presence of at least one molecular species from the capture substrate;
f) loading the capture substrate from the second minienvironment into a transfer container, wherein the transfer container also holds at least one electronic substrate loaded from the second minienvironment;
g) transporting the transfer container from the second minienvironment to a third minienvironment;
h) removing the capture substrate from the transfer container; and
i) analyzing the capture substrate for the presence of at least one molecular species.

57. The method of claim 55, wherein the electronic substrate is a silicon wafer and the capture substrate comprises a silicon surface having a surface area greater than the silicon wafer.

58-59. (canceled)

60. The method of claim 55, wherein analyzing the capture substrate includes desorbing at least one molecular species from the capture substrate.

61. A method of determining the presence of a molecular species in a transfer container operating in an electronics manufacturing process, comprising:
a) completing at least one processing step in an electronics manufacturing process having a plurality of steps;
b) loading a capture substrate into a transfer container, wherein the transfer container also holds at least one electronic substrate processed during the at least one processing step;
c) transporting the transfer container to a location to perform a subsequent processing step;
d) removing the capture substrate and at least one electronic substrate from the transfer container;
e) analyzing the capture substrate to determine the presence of the molecular species;
f) optionally completing at least one additional processing step and repeating steps b), c), d), and e).

62-65. (canceled)
